(19) United States
(12) Patent Application Publication
(10) Pub. No.: US 2024/0108343 A1
(43) Pub. Date: Apr. 4, 2024

(54) SURGICAL INSTRUMENT WITH LOCKOUT MECHANISM

(71) Applicant: Intuitive Surgical Operations, Inc., Sunnyvale, CA (US)

(72) Inventor: Matthew Wixey, San Jose, CA (US)

(73) Assignee: Intuitive Surgical Operations, Inc., Sunnyvale, CA (US)

(21) Appl. No.: 18/531,313

(22) Filed: Dec. 6, 2023

Related U.S. Application Data
(63) Continuation of application No. 17/974,627, filed on Oct. 27, 2022, now Pat. No. 11,864,762.
Application No. 17/974,627 is a continuation of application No. 16/966,099, filed on Jul. 30, 2020, now Pat. No. 11,517,312.
(60) Provisional application No. 62/629,572, filed on Feb. 12, 2018.

(51) Int. Cl. A61B 17/072; A61B 17/28

(52) U.S. Cl. CPC A61B 17/07207; A61B 17/2841; A61B 2017/07271; A61B 2017/07278; A61B 2017/07285; A61B 2017/2845

(57) ABSTRACT

Surgical instruments, such as clamping and cutting instruments, are provided. In one aspect, a surgical stapling instrument comprises first and second jaws and a drive member configured to move in a distal direction through at least one of the jaws. The instrument further includes a locking member movable between a first position permitting distal translation of the drive member, and a second position impeding distal translation of the drive member, and a switch movable from a proximal position to a distal position. When the switch is in the proximal position, it releasably maintains the locking member in the first position, and when the switch is in the distal position, it allows the locking member to move to the second position. This inhibits or prevents actuation of a knife when there is a spent or previously fired cartridge loaded in the instrument.

### CROSS REFERENCE TO RELATED APPLICATIONS

This application is a Continuation of U.S. Nonprovisional application Ser. No. 17/974,627, filed Oct. 27, 2022, which is a Continuation of U.S. Nonprovisional application Ser. No. 16/966,099, filed Jul. 30, 2020, which is the U.S. National Stage of International Application No. PCT/US19/17646 filed Feb. 12, 2019 which claims the benefit of U.S. Provisional Application No. 62/629,572 filed Feb. 12, 2018, the entire disclosures of which are incorporated by reference for all purposes.

### TECHNICAL FIELD

The present description relates to surgical stapling instruments having a locking mechanism to prevent actuation of a knife when there is a spent or previously fired cartridge in place. More particularly, the present description is directed towards locking assemblies for surgical clamping and cutting instruments include a locking member and a switch.

### BACKGROUND

Surgical clamping and cutting instruments, such as, for example, surgical stapling instruments, may include an end effector having opposing jaws that clamp tissue and a knife that cuts the clamped tissue. It is often advantageous for an end effector of a surgical stapling instrument to be reusable. To that end, staple cartridges can be fitted into one jaw of the end effector prior to each use of the surgical stapling instrument.

It is desirable to prevent firing of a surgical stapling instrument while a spent cartridge remains in place on the jaw. Thus, a need exists for effective mechanisms to prevent firing of a surgical stapling instrument while a spent staple cartridge is in place in the end effector of the surgical stapling instrument.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present description relates to surgical stapling instruments having a locking mechanism to prevent actuation of a knife when there is a spent or previously fired cartridge in place.

In one aspect, a lockout assembly for use with a surgical stapling instrument includes a locking member and a switch. A drive member is configured to releasably engage and translate at least one of a knife or a shuttle in a distal direction through a staple firing stroke. The locking member of the locking assembly is movable from a first position permitting distal translation of the drive member through the staple firing stroke, to a second position inhibiting distal translation of the drive member through the staple firing stroke. A spring may bias the locking member toward the second position. The switch of the locking assembly is movable from a proximal position to a distal position. When the switch is in the proximal position, the switch releasably maintains the locking member in the first position. When the switch is in the distal position, the switch disengages from the locking member thereby allowing the locking member to move to the second position.

In another aspect, a surgical stapling instrument includes an anvil jaw assembly, and a staple jaw assembly, including a knife and a shuttle. A drive member is configured to releasably engage and translate the knife and shuttle in a distal direction through a staple firing stroke. The knife and shuttle disengage from the drive member upon distal movement of the drive member after the staple firing stroke. The surgical stapling instrument further includes a locking member pivotable from a first position permitting distal translation of the drive member, to a second position preventing distal translation of the drive member. The surgical stapling instrument further includes a switch, that when in a proximal position, releasably maintains the locking member in the first position. When the switch is in a distal position, the switch disengages from the locking member thereby allowing the locking member to move to the second position.

In another aspect, a surgical stapling instrument includes an anvil jaw assembly, and a staple jaw assembly including a shuttle. The surgical stapling instrument further includes a drive member having a knife integrally formed on an edge thereof. The drive member is configured to releasably engage and translate the shuttle in a distal direction through a staple firing stroke. The shuttle disengages from the drive member upon subsequent distal movement of the drive member after the staple firing stroke. The surgical stapling instrument further includes a locking member pivotable from a first position permitting distal translation of the drive member, to a second position preventing distal translation of the drive member. The surgical stapling instrument further includes a switch, that when in a proximal position, releasably maintains the locking member in the first position. When the switch is in a distal position, the switch disengages from the locking member thereby allowing the locking member to move to the second position.

### BRIEF DESCRIPTION OF THE DRAWINGS

The above and other aspects, features, and advantages of the present surgical stapling instruments having a locking mechanism will become more apparent in light of the following detailed description when taken in conjunction with the accompanying drawings in which:

FIG. 1A is a perspective view of an illustrative surgical stapling instrument;

FIG. 1B is an exploded view of an illustrative end effector of a surgical stapling instrument;

FIG. 2 depicts a partial cross-sectional side view of the end effector of a surgical stapling instrument including a lockout assembly in accordance with an embodiment having an unfired reload installed;

FIG. 3 is a partial perspective view illustrating the end effector including a lockout assembly in accordance with the embodiment of FIG. 2 showing a proximally positioned switch of a surgical stapling instrument having an unfired reload is installed;

FIG. 3a is a partial perspective view illustrating the end effector including a lockout assembly in accordance with the embodiment of FIG. 2 illustrating a pair of retaining snaps of the switch engaging undercuts of the unfired reload;

FIG. 4 is a partial cross-sectional side view of the end effector including a lockout assembly in accordance with the embodiment of FIG. 2 with the drive member advanced to contact the knife;

FIG. 5 is a partial side, cross-sectional view of the end effector including a lockout assembly in accordance with the embodiment of FIG. 2 illustrating the switch advanced to a distal position;

FIG. 6 is a partial side, cross-sectional view of the end effector including a lockout assembly in accordance with the embodiment of FIG. 2 showing the knife cutting the switch;

FIG. 6a is a partial perspective view of the end effector including a lockout assembly in accordance with the embodiment of FIG. 2 showing a distal wall blocking the switch from translating distally;

FIG. 7 is a partial perspective view of the end effector including a lockout assembly in accordance with the embodiment of FIG. 2 showing an uncut switch that has been pushed to a distal position by the knife;

FIG. 8 is a partial perspective view of the end effector including a lockout assembly in accordance with the embodiment of FIG. 2 having a cut switch that is prevented from retracting by a pair of stops;

FIG. 9 is a partial cross-sectional side view of the end effector including a lockout assembly in accordance with the embodiment of FIG. 2 with the shuttle shown; and

FIG. 10 is a partial cross-sectional side view of the end effector including a lockout assembly in accordance with the embodiment of FIG. 2 illustrating a drive member having an integrally formed knife on an edge thereof.

### DETAILED DESCRIPTION

Particular embodiments of the present surgical stapling instruments are described hereinbelow with reference to the accompanying drawings; however, it is to be understood that the disclosed embodiments are merely exemplary of the description and may be embodied in various forms. Therefore, specific structural and functional details disclosed herein are not to be interpreted as limiting, but merely as a basis for the claims and as a representative basis for teaching one skilled in the art to variously employ the present description in virtually any appropriately detailed structure. Well-known functions or constructions are not described in detail to avoid obscuring the present description in unnecessary detail.

The present description relates to locking assemblies including a locking member and a switch. A drive member is configured to engage at least one of a knife or a shuttle of a surgical stapling instrument and to translate the knife and/or shuttle in a distal direction through a staple-firing stroke. Contact between the drive member and the knife and/or shuttle is releasable in that once the knife and/or shuttle are translated by the drive member in the distal direction through a staple firing stroke, the knife and/or shuttle disengage from the drive member, remain at a distal portion of the stapling instrument, and are not translated in a proximal direction by the drive member. The locking member is movable from a first position permitting distal translation of the drive member through the staple-firing stroke, and a second position inhibiting distal translation of the drive member through the staple-firing stroke. A spring is configured to bias the locking member toward the second position. The switch of the locking assembly is movable from a proximal position to a distal position. When the switch is in the proximal position, the switch releasably maintains the locking member in the first position. When the switch is in the distal position, the switch disengages from the locking member thereby allowing the locking member to move to the second position.

While the following description is presented with respect to a linear surgical stapler where staples are sequentially fired, it should be understood that the present locking assemblies may be readily adapted for use in any type of surgical clamping and cutting instruments, whether or not the surgical clamping and cutting instrument applies a fastener. The surgical clamping and cutting instrument may be a minimally invasive (e.g., laparoscopic) instrument or an instrument used for open surgery.

Additionally, the present locking assemblies may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

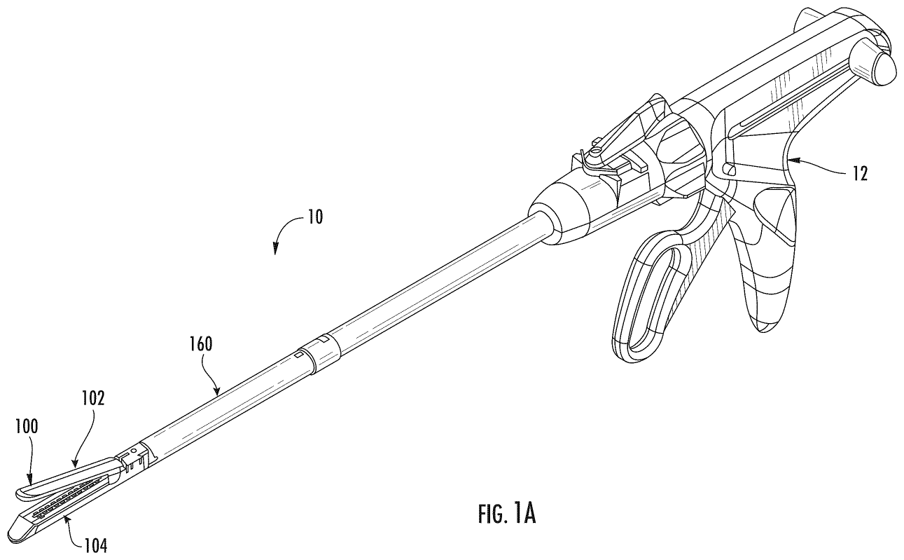

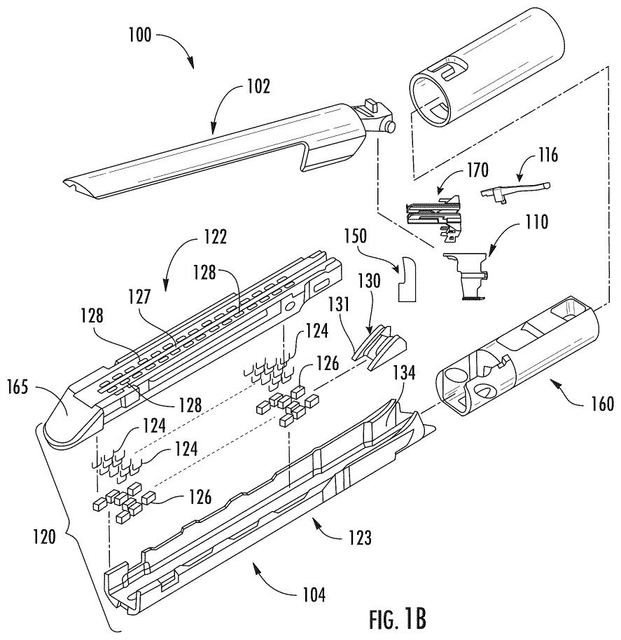

FIG. 1A is a perspective view of an illustrative surgical stapling instrument 10 capable of utilizing a locking assembly. Surgical stapling instrument 10 includes a handle assembly 12, and an end effector 100 including an anvil jaw assembly 102 and a staple jaw assembly 104 mounted on an elongated shaft 160 of the surgical stapling instrument 10.

FIG. 1B shows anvil jaw assembly 102, including an anvil 106 having staple forming pockets 103 (not shown) supported thereon, and staple jaw assembly 104. Staple jaw assembly 104 and anvil jaw assembly 102 are configured to move from an open position to a closed position. In the open position, a fresh stapling cartridge can be loaded into jaw assembly 104, a spent staple cartridge removed from jaw assembly 104, and tissue may be positioned between the jaw assemblies 102, 104. In the closed position, jaw assemblies 102, 104 cooperate to close upon and clamp tissue such that cartridge 122 and anvil 106 are in close cooperative alignment. In the embodiment shown in FIGS. 1A and 1, staple jaw assembly 104 is stationary and anvil jaw assembly 102 pivots to the open position. In other embodiments it is contemplated that the jaw assembly containing the anvil is stationary and the jaw assembly containing the staple cartridge pivots to the open position. As those skilled in the art reading this description will appreciate, in yet other embodiments both the anvil jaw assembly and the staple jaw assembly may pivot.

With continued reference to FIG. 1B, staple jaw assembly 104 includes a staple cartridge 122 supported in a channel 134 on a lower jaw 123. Cartridge 122 includes a plurality of staples 124 that are supported on corresponding staple drivers 126 provided within respective staple apertures 128 formed in cartridge 122. Cartridge 122 also includes a shuttle 130 having an inclined distal portion 131 that, upon distal movement, sequentially acts on staple drivers 126, camming them upwardly thereby moving staples 124 into deforming contact with anvil 106. Cartridge 122 also includes a knife 150 configured to translate distally through a channel 127 in cartridge 122 and sever clamped, stapled tissue.

FIG. 1B further shows a drive member 110 that is movably supported on the surgical stapling instrument such that it may pass distally through cartridge 122 and staple jaw assembly 104 when the surgical stapling instrument is fired (e.g., actuated). Also shown in FIG. 1B is the locking assembly including locking member 116 on staple jaw assembly 104 and switch 170 on cartridge 122.

For a more detailed description of illustrative end effectors, reference may be made to U.S. Pat. Nos. 6,669,073 and 8,800,841, the entire contents of which are incorporated herein by this reference. It should of course, be understood that end effector shown in FIGS. 1A and 1B is merely illustrative, and that other end effectors may be employed, including but not limited to the end effectors shown in WO2014/106275, the entire contents of which are incorporated herein by this reference.

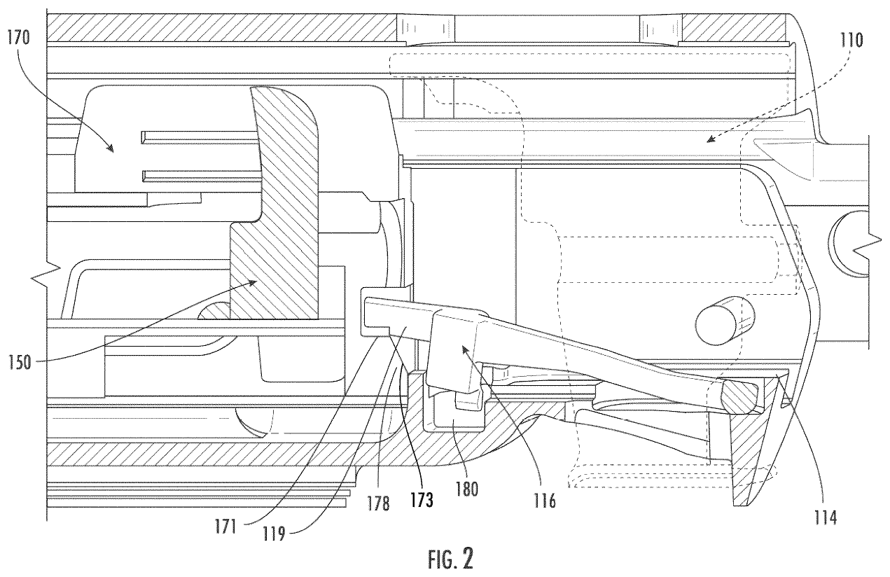

FIG. 2 shows an illustrative surgical stapling instrument with an unfired reload installed, including drive member 110, spring 114, locking member 116, knife 150, switch 170, and slot 180.

In a fresh reload, drive member 110 is in a proximal position where it has not yet engaged knife 150. Drive member 110 may be any structure capable of pushing at least one of a shuttle or a knife of a surgical stapling instrument with the necessary force to effectively sever or staple human tissue. Drive member 110 may be an I-beam, an E-beam, or any other type of drive member capable of performing similar functions. Drive member 110 includes a lower distal portion 111 and upper distal portion 113.

When an unfired reload is installed, as in FIG. 2, switch 170 is in a first proximal position. In a fresh, unfired reload distal portion 119 of locking member 116 rests on shelf 171 of switch 170, keeping engagement portion 118 of locking member 116 above and out of engagement with slot 180. When locking member 116 is in this disabled position, distal translation of drive member 110 is permitted, as locking member 116 will not obstruct movement of drive member 110.

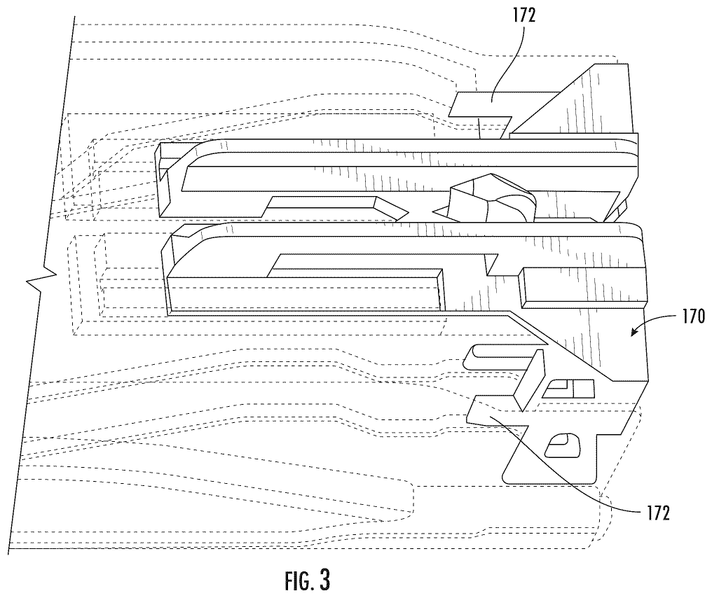

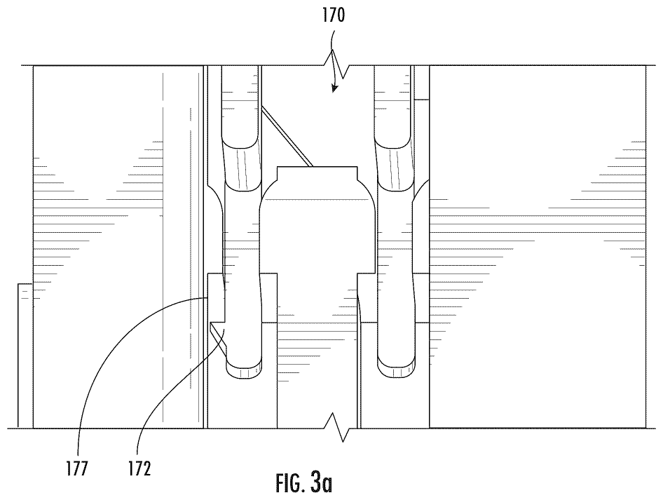

As seen in FIG. 3, an unfired reload containing a new cartridge, knife 150 is located on the proximal side of central portion 174 of switch 170, which is uncut. Upon installation of an unfired reload, as depicted in FIG. 3a, retaining snaps 172 of switch 170 are configured to engage undercuts 177 formed on the unfired reload. This interaction, which may be a snap-fit interaction, retains the switch in the proximal position prior to firing of the surgical stapling instrument.

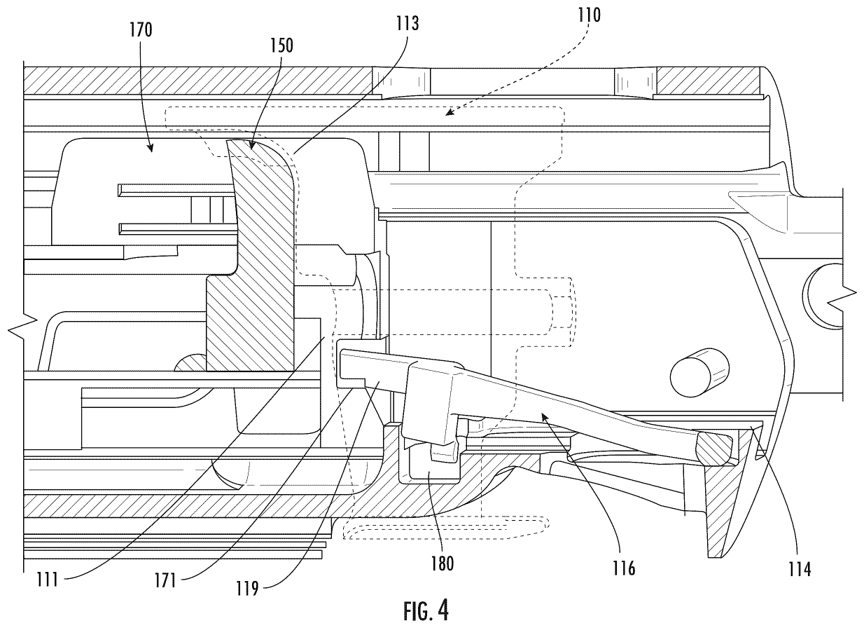

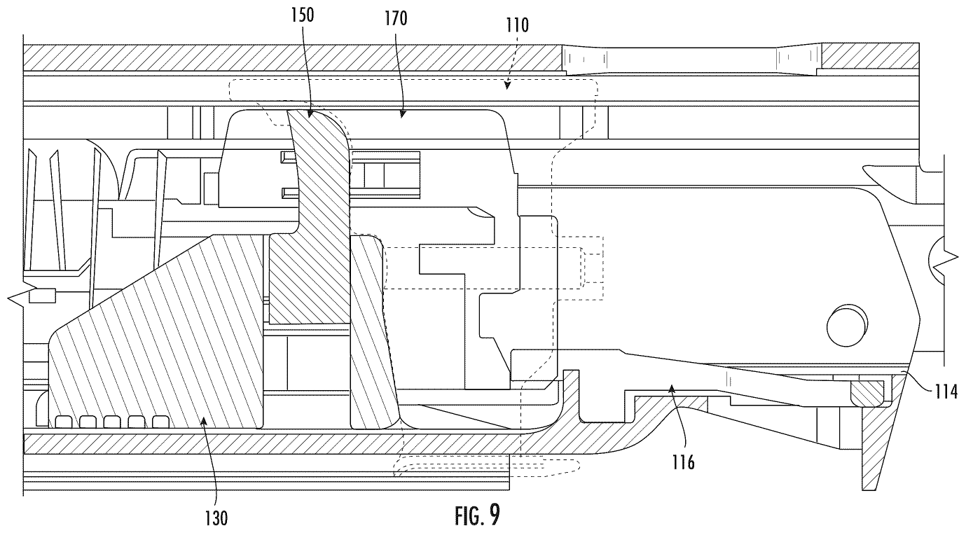

Upon initiation of the staple-firing stroke, as seen in FIG. 4 drive member 110 moves distally to contact knife 150. At this point in the actuation stoke, locking member 116 remains out of engagement with slot 180 because distal portion 119 of the locking member continues to rest on shelf 171 of switch 170. In this position, upper distal portion 113 of drive member 110 contacts knife 150 for distal translation as the actuation stroke continues. Lower distal portion 111 of drive member 110 similarly moves distally in order to engage and translate shuttle 130 (see FIG. 9).

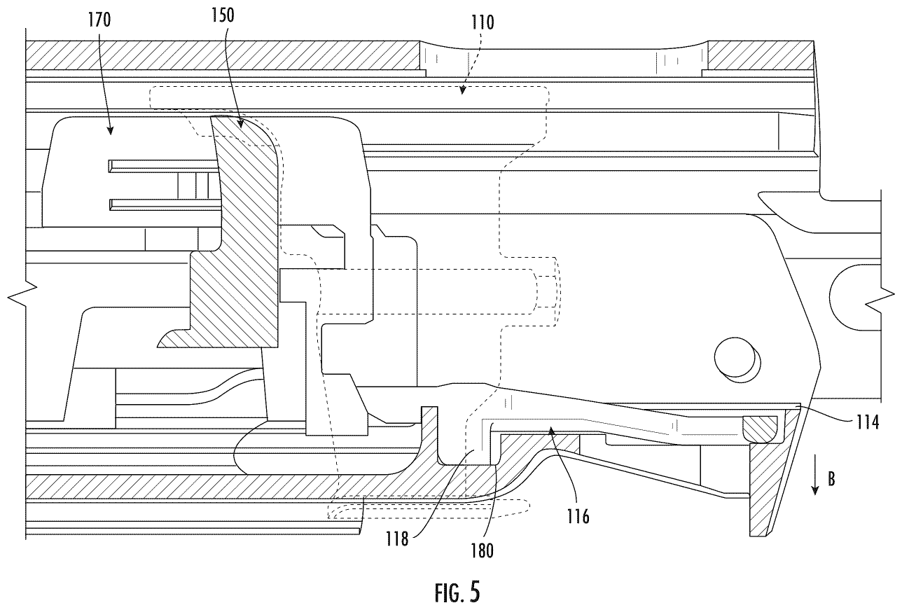

As illustrated in FIG. 5, as drive member 110 continues to move distally, knife 150 begins to advance. Knife 150 is in contact with, and therefore pushes and translates, switch 170 from a proximal position, to a distal position. As illustrated in FIG. 5, once in the distal position, switch 170 separates from and no longer supports distal portion 119 of locking member 116 which essentially falls off of shelf 171. As a result, engagement portion 118 of locking member 116 falls into and engages slot 180, enabling the lockout. Spring 114 is configured to bias engagement portion 118 of locking member 116 in the direction of Arrow “B”, urging engagement portion 118 to drop into slot 180 to enable the locking mechanism.

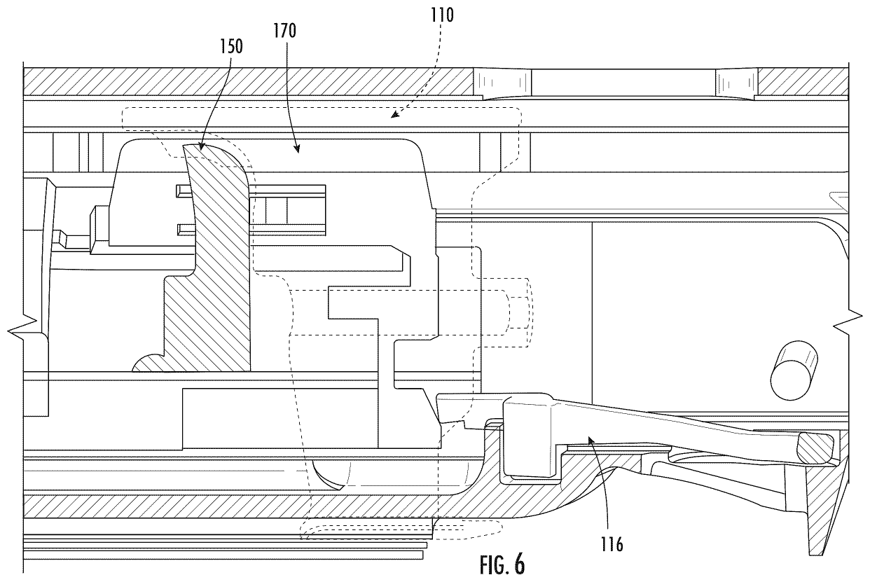

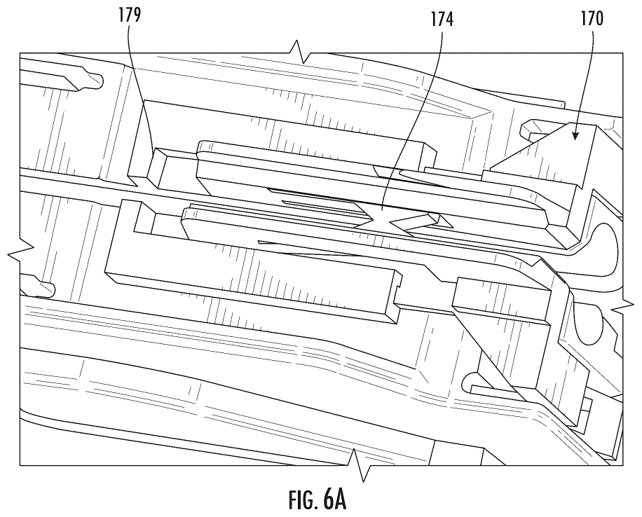

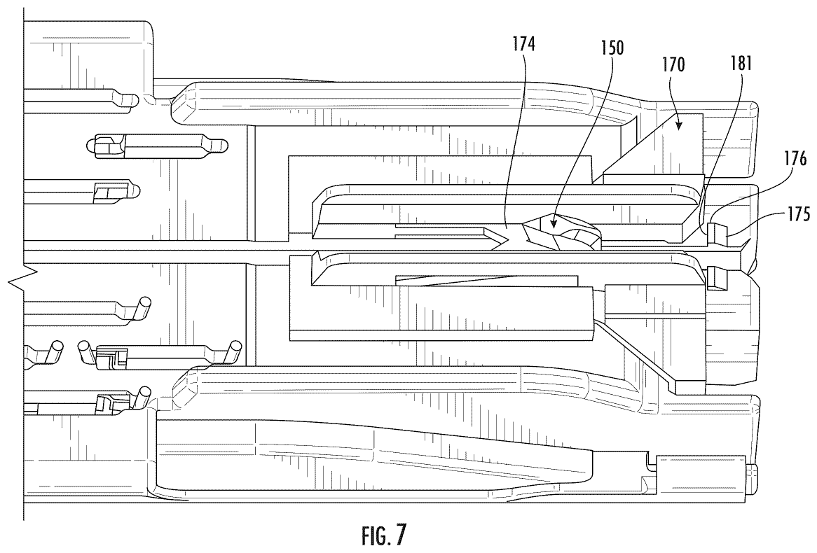

In FIG. 6, switch 170 is in the distal position. Once switch 170 is translated distally, further distal translation is prevented as switch 170 becomes obstructed by distal wall 179 as best seen in FIG. 6a. As a result of this obstruction, continued distal translation of knife 150 cuts through central portion 174 of switch 170 (see FIG. 7), as a result of the force provided by drive member 110 translating knife 150 distally. A perspective view of switch 170 in the second distal position is shown in FIG. 7 just before cutting of central portion 174. FIG. 7 also illustrates stops 176, each including a proximal ramped face 175. As drive member 110 translates knife 150 distally, and knife 150 pushes switch 170 distally, switch 170 rides over ramped faces 175 of stops 176. Distal faces 181 of stops 176 prohibit movement of switch 170 back to its initial proximal position.

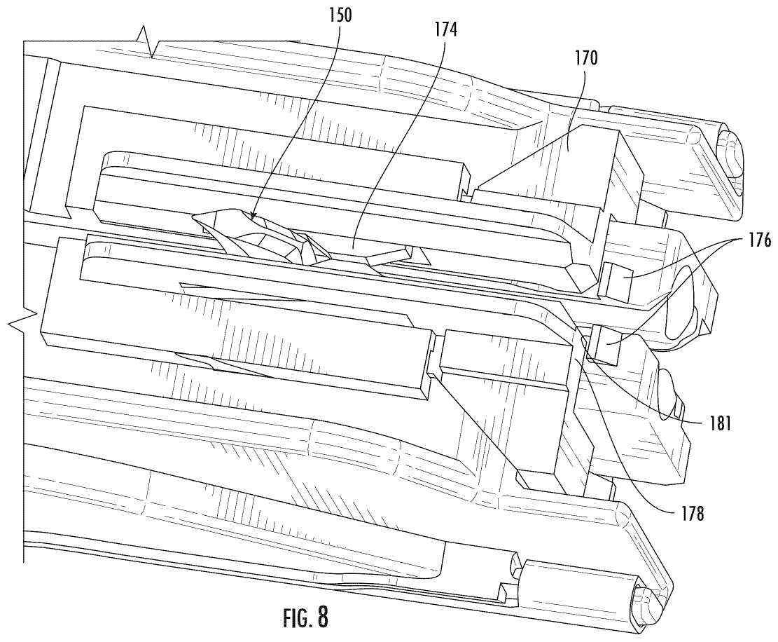

In FIG. 8, knife 150 has cut through central portion 174 of switch 170 so that knife 150 may continue to translate distally towards a final, parked position. As illustrated, stops 176 are positioned on the proximal side of switch 170. In this position, retraction of switch 170 is impossible, as the proximal portions 178 of switch 170 abut distal faces 181 of stops 176. By preventing switch 170 from retracting, it is ensured that locking member 116 may not be disengaged, while also keeping both cut parts of switch 170 contained in the reload.

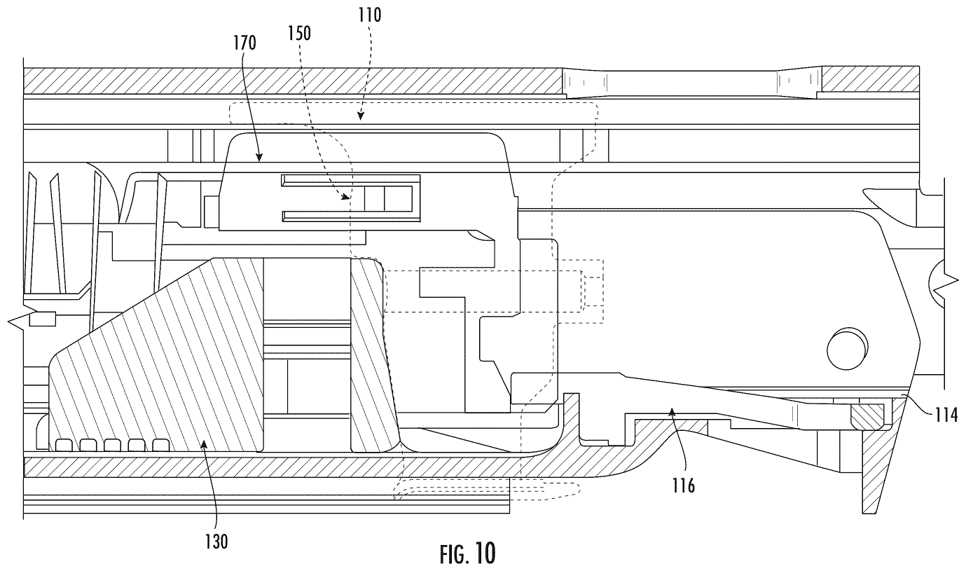

Once drive member 110 translates distally through a complete firing stroke during which stapling and severing of tissue have occurred, drive member 110 can be retracted, leaving knife 150 parked at a position in a distal portion of cartridge 122. In embodiments, such as the embodiment illustrated in FIG. 9 a shuttle 130 may be unable to move proximally towards the home position due to friction with cartridge 122. In embodiments, as illustrated in FIG. 10, knife 150 may be integrally formed with drive member 110. In embodiments, knife 150 may be parked in a predetermined position in a distally located garage 165. The garage 165 including lateral surfaces that face the cutting tip of knife 150. As drive member 110 is retracted, engagement portion 118 of locking member 116 is unable to move upwards out of slot 180, as spring 114 retains it in the locked position.

Further retraction of drive member 110 positions locking member 116 distal of the drive member. Because the staple cartridge is spent and there is no proximally positioned switch to hold the locking member out of engagement with slot 180, any attempt to re-fire the surgical stapling instrument will be prevented by drive member 110 engaging locking member 116.

In order to disable the lockout of the surgical stapling instrument, an unfired reload (i.e. a new cartridge) must be installed. Each new cartridge contains a new switch 170, shuttle 130, and knife 150. When a new cartridge is installed, switch 170 moves proximally causing a distal portion 119 of locking member 116 to engage with and ride upwards along proximal ramped surface 173 of switch 170, until locking member 116 is again resting on shelf 171, as may be seen in FIG. 2. The lockout is then disabled, allowing for a user to again fire the surgical stapling instrument.

While several embodiments of the description have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A surgical stapling instrument comprising:
a shaft having a longitudinal axis and an end effector with first and second jaws;
a drive member configured to move in a distal direction through at least one of the jaws;
a locking member movable between a first position permitting distal translation of the drive member, and a second position impeding distal translation of the drive member;
a switch movable from a proximal position to a distal position, wherein when the switch is in the proximal position the switch releasably maintains the locking member in the first position, and wherein when the switch is in the distal position the switch allows the locking member to move to the second position; and
a surface extending laterally through a portion of the end effector and positioned to prevent distal movement of the switch.

2. The surgical stapling instrument of claim 1, wherein the surface comprises a blocking wall extending laterally through the end effector and positioned to prevent further distal movement of the switch when the switch is in the distal position.

3. The surgical stapling instrument of claim 2, wherein the end effector comprises an internal channel for receiving a knife on the drive member, wherein the blocking wall comprises first and second walls disposed laterally outward from the internal channel.

4. The surgical stapling instrument of claim 1, further comprising a stop member within the end effector, the stop member configured to inhibit proximal movement of the switch.

5. The surgical stapling instrument of claim 4, wherein the stop member comprises an inclined surface that allows distal movement of the switch to the distal position and prevents proximal movement of the switch from the distal position.

6. The surgical stapling instrument of claim 1, wherein the switch disengages from the locking member when moved to the distal position.

7. The surgical stapling instrument of claim 1, wherein the locking member engages a slot in one of the first and second jaws when in the second position.

8. The surgical stapling instrument of claim 1, wherein the drive member is configured to pass through a portion of the switch as the drive member is moved in the distal direction through one of the jaws.

9. The surgical stapling instrument of claim 1 further comprising a spring configured to bias the locking member towards the second position.

10. The surgical stapling instrument of claim 1, wherein the drive member is actuated by a control device of a robotic surgical system.

11. A surgical stapling instrument comprising:
a shaft having a longitudinal axis and an end effector with first and second jaws;
a drive member configured to move in a distal direction through at least one of the jaws;
a locking member movable between a first position permitting distal translation of the drive member, and a second position impeding distal translation of the drive member;
a switch movable from a proximal position to a distal position, wherein when the switch is in the proximal position the switch releasably maintains the locking member in the first position, and wherein when the switch is in the distal position the switch allows the locking member to move to the second position; and
a stop member within the end effector configured to inhibit proximal movement of the switch.

12. The surgical stapling instrument of claim 11, wherein the stop member comprises an inclined surface that allows distal movement of the switch to the distal position and prevents proximal movement of the switch from the distal position.

13. The surgical stapling instrument of claim 12, wherein the inclined surface extends away from the longitudinal axis in the distal direction and the stop member comprises a distal face that prevents proximal movement of the switch from the distal position.

14. The surgical stapling instrument of claim 11, further comprising a surface extending laterally through a portion of the end effector and positioned to prevent distal movement of the switch.

15. The surgical stapling instrument of claim 14, wherein the surface comprises a blocking wall extending laterally through the end effector and positioned to prevent further distal movement of the switch when the switch is in the distal position.

16. The surgical stapling instrument of claim 15, wherein the end effector comprises an internal channel for receiving a knife on the drive member, wherein the blocking wall comprises first and second walls disposed laterally outward from the internal channel.

17. The surgical stapling instrument of claim 11, wherein the drive member is actuated by a control device of a robotic surgical system.

18. A surgical stapling instrument comprising:
a shaft having a longitudinal axis and an end effector with first and second jaws;
a drive member configured to move in a distal direction through at least one of the jaws;
a locking member movable between a first position permitting distal translation of the drive member, and a second position impeding distal translation of the drive member;
a switch movable from a proximal position to a distal position, wherein when the switch is in the proximal position the switch releasably maintains the locking member in the first position, and wherein when the switch is in the distal position the switch allows the locking member to move to the second position, wherein the end effector is configured to prevent the switch from moving distally of the distal position.

19. The surgical stapling instrument of claim 18, wherein the end effector is configured to prevent the switch from moving from the distal position to the proximal position.
